(19) United States
(12) Patent Application Publication
(10) Pub. No.: US 2008/0305095 A1
(43) Pub. Date: Dec. 11, 2008

(54) Nutritional Drink

(76) Inventor: Mitchell Karl, Boca Raton, FL (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(21) Appl. No.: 12/120,765

(22) Filed: May 15, 2008

Related U.S. Application Data
(60) Provisional application No. 60/942,122, filed on Jun. 5, 2007.

(51) Int. Cl. A61K 31/122; A61K 36/06; A61K 31/205; A61K 31/375; A61K 31/455; A61K 36/00; A61P 3/06; A61P 9/10

(52) U.S. Cl. 424/94.1; 424/195.16; 424/725; 514/556; 514/474; 514/356

(57) ABSTRACT

A first composition of drink ingredients specifically designed to lower cholesterol that addresses multiple mechanisms including hepatic synthesis and release, intestinal absorption of cholesterol, while, at the same time, including ingredients that mitigate the side effects of the constituents and increase their efficacy by affecting emotional factors that influence compliance such as a sense of well-being and euphoria on the one hand, or an increased overall metabolism and desire for the product stemming from its coloration on the other hand.

### CROSS-REFERENCE TO RELATED APPLICATION

This application claims the priority of U.S. provisional patent application Ser. No. 60/942,122 filed on Jun. 5, 2007.

### FIELD OF THE INVENTION

This invention relates to nutritional supplements. More particularly, the invention relates to compositions and methods for supplementing the diet for improving health and preventing disease.

### BACKGROUND

Chronically elevated blood levels of cholesterol lead to cardiovascular disease as the cholesterol finds its way into the walls of blood vessels and damages them. This ultimately results in symptoms of chronic arterial insufficiency such as angina and claudication on the one hand, and acute vascular insufficiency, such as heart attack and stroke on the other.

Over $120 billion dollars is spent on direct and indirect costs associated with cardiovascular disease annually in the United States alone. Cardiovascular disease incidence increases with serum LDL cholesterol in a log linear fashion and more importantly declines with treatment-induced reduction of serum LDL cholesterol.

Conventional therapy for elevated blood cholesterol levels takes the form of four classes of FDA approved medications: statins, bioresins, fibrates, and niacin.

Of these, statins are the most widely used with greater than $20 billion in annual sales; however, all the classes, including statins, have side effects and at higher doses, that are necessary to achieve targets, result in side effects that limit their utility. This is especially so of niacin and statins.

Recently, it has been appreciated that in attempting to lower cholesterol, two or more drugs with different mechanisms of action can lower toxicity and produce synergy in the cholesterol lowering effect. Vytorin, a recently introduced combination of Zetia and Simvastatin, has been shown to decrease cholesterol absorption and synthesis and reduce cholesterol better than the sum of the expected reduction of either drug alone. This is explained by a phenomenon I refer to as “escape homeostasis.” When one pathway to cholesterol elevation is blocked, an alternative pathway is often enhanced by the body, so that the overall cholesterol levels are maintained. There is, thus, a built-in or automatic drug resistance that can only be overcome with multiple active agents working simultaneous at different sites. It is noteworthy that evidence exists that even homeopathic, previously felt sub-therapeutic amounts of biologically active cholesterol lowering compounds can exert powerful efficacy with minimal side effects when combined with other agents that work by alternative pathways.

### SUMMARY

The invention is based on the development of a cholesterol lowering beverage that utilizes multiple mechanisms and sites of action to reduce cholesterol and also has ingredients that reduce toxicity, rev up metabolism both on a general and a cellular level and, finally stimulate compliance by causing a sense mild euphoria and well-being, as well as enhancing metabolism.

Accordingly, in one aspect, the invention features a nutritional beverage in a container. The beverage includes water and at least two (2, 3, 4, 5 or 6) of niacin, a phytosterol component, red yeast rice, coenzyme Q10, L-carnitine, and ascorbic acid. The beverage in the container can include between about 50 and 100 mg of niacin, between about 700 and 2000 mg of the phytosterol component, between about 1200 and 2400 mg of red yeast rice, between about 200 and 1000 mg of L-carnitine, between about 100 and 2000 mg of ascorbic acid, and/or the drink product of claim 1, wherein the beverage in the container comprises between about 10 and 200 mg of coenzyme Q10.

In another aspect, the invention features a method for lowering serum cholesterol levels in a subject. This method includes the step of administering to the subject the drink product described above.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The invention provides an aqueous composition of ingredients and dietary supplements to yield a cholesterol lowering drink. The composition is typically contained within a single serving container such as a can or bottle and includes water and a combination of at least two of niacin, a phytosterol component, red yeast rice, coenzyme Q10, L-carnitine, and ascorbic acid in amount effective to reduce serum cholesterol levels in a subject who drinks the composition on a regular basis (e.g., once a day, twice a day, every two days, or every three days).

The active ingredients are preferably included at a concentration effective to reduce a subject's serum cholesterol by at least 10% (e.g., at least 20, 30, 40, or 50%) when included in combination with each other and administered to the subject on a regular basis (e.g., twice a day or once a day for at least 2, 3, 4, 5, 6, 12, or 24 weeks).

Niacin lowers cholesterol by inhibiting lipoprotein formation or release in the liver. Phytosterols lower cholesterol by competing for absorption in the intestines. Red Yeast Rice is a dietary supplement and coloring agent that has been used in Chinese food and medicines for centuries. It decreases synthesis and absorption of cholesterol, produces red coloration which stimulates appetite, compliance, and metabolism. On a cellular level, L-carnitine and coenzyme Q10 enhance transport of fatty acids to the mitochondria and enhance the burning of fatty acids respectively. L-carnitine can also cause a mild sense of euphoria, a beneficial effect that may result in greater compliances with the preparation containing it. Ascorbic acid changes the constitution of bile to decrease cholesterol absorption and inhibit the HMG-CoA reductase pathway, the rate-limiting step in cholesterol biosynthesis.

### EXAMPLE

A beverage for once a day administration includes water (100-500 ml) and phytosterols (between about 700 and 2000 mg), niacin (between about 50 to 100 mg), red yeast rice (between about 1200 to 2400 mg), coenzyme Q10 (between about 10-200 mg; e.g., about 50 mg), ascorbic acid (vitamin C; between about 100 and 2000 mg), and L-carnitine (between about 200 and 1000 mg).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nutritional beverage in a container, the beverage comprising water and at least two active ingredients selected from the group consisting of niacin, a phytosterol component, red yeast rice, coenzyme Q10, L-carnitine, and ascorbic acid.

2. The drink product of claim 1, wherein the beverage in the container comprises between about 50 and 100 mg of niacin.

3. The drink product of claim 1, wherein the beverage in the container comprises between about 700 and 2000 mg of the phytosterol component.

4. The drink product of claim 1, wherein the beverage in the container comprises between about 1200 and 2400 mg of red yeast rice.

5. The drink product of claim 1, wherein the beverage in the container comprises between about 200 and 1000 mg of L-carnitine.

6. The drink product of claim 1, wherein the beverage in the container comprises between about 100 and 2000 mg of ascorbic acid.

7. The drink product of claim 1, wherein the beverage in the container comprises between about 10 and 200 mg of coenzyme Q10.

8. The drink product of claim 1, wherein the beverage comprises at least three active ingredients selected from the group consisting of niacin, a phytosterol component, red yeast rice, coenzyme Q10, L-carnitine, and ascorbic acid.

9. The drink product of claim 1, wherein the beverage comprises at least four active ingredients selected from the group consisting of niacin, a phytosterol component, red yeast rice, coenzyme Q10, L-carnitine, and ascorbic acid.

10. The drink product of claim 1, wherein the beverage comprises at least five active ingredients selected from the group consisting of niacin, a phytosterol component, red yeast rice, coenzyme Q10, L-carnitine, and ascorbic acid.

11. The drink product of claim 1, wherein the beverage comprises niacin, a phytosterol component, red yeast rice, coenzyme Q10, L-carnitine, and ascorbic acid.

12. A nutritional beverage in a container, the beverage comprising water, about 50 and 100 mg of niacin, between about 700 and 2000 mg of phytosterol component, between about 1200 and 2400 mg of red yeast rice, between about 10 and 200 mg of coenzyme Q10, between about 200 and 1000 mg of L-carnitine, and between about 100 and 2000 mg ascorbic acid.

13. A method comprising the step of administering to the subject the drink product of claim 1 at least once a day for at least 7 days.
